(19) United States
(12) Patent Application Publication
(10) Pub. No.: US 2014/0357726 A1
(43) Pub. Date: Dec. 4, 2014

(54) USE OF PEGYLATED ALCOHOLS FOR THE TREATMENT OF ACTINIC KERATOSIS

(71) Applicant: MERZ PHARMA GmbH & CO. KGaA, FRANKFURT am MAIN (DE)

(72) Inventors: Heiko Barg, Weinheim (DE); Rainer Pooth, Bad Soden/Taunus (DE); Harry Frank Abts, Oberursel (DE); Kevin Kiehm, Telgte (DE)

(21) Appl. No.: 14/361,112

(22) PCT Filed: Nov. 29, 2012
(86) PCT No.: PCT/EP2012/004943

§ 371 Date: May 28, 2014

Related U.S. Application Data
(60) Provisional application No. 61/564,893, filed on Nov. 30, 2011.

(30) Foreign Application Priority Data
Nov. 30, 2011 (EP) 11191387.7

(51) Int. Cl. A61K 31/08

(52) U.S. Cl. CPC A61K 31/08
USPC 514/723

(57) ABSTRACT

Use of pegylated alcohols for the treatment of actinic keratosis.

The present invention relates to the use of pegylated alcohols for the treatment of actinic keratosis.

In general, actinic keratosis (also called “solar keratosis” and “senile keratosis”, AK) is a premalignant condition of thick, scaly, or crusty patches of skin consisting of dysplastic keratinocytic lesions. AK is one of the most common conditions treated by dermatologists. It is more common in fair-skinned people. And most important it is associated with those who are frequently exposed to the sun, as it is usually accompanied by solar damage. It is generally accepted that these lesions can progress to squamous cell carcinoma (SSC). Concerning the rate of this transformation there is a controversy in the literature. Annual rates of transformation are ranging from 0.1% -20%. Nevertheless there is no doubt that these pre-cancerous lesions should be treated in order to prevent manifestation of malignant tumors. In addition lesions are in general treated also for cosmetic purposes and to provide relief from symptoms, such as tenderness or itch.

When skin is exposed to the sun constantly, thick, scaly, or crusty bumps may appear. The scaly or crusty part of the bump is dry and rough. The growths start out as flat scaly areas, and later grow into a tough, wart-like area.

In addition to chronic UV-exposure also infections with HPV has been implicated in the aetiology of AK.

An actinic keratosis site commonly ranges between 2 and 6 millimeters in size, and can be dark or light, tan, pink, red, a combination of all these, or have the same pigment as the surrounding skin. It typically appears on any sun-exposed area, such as the face, ears, neck, scalp, chest, backs of hands, forearms, or lips.

An efficient treatment that can be used to treat larger areas of affected skin and eliminate obvious AK lesions as well as clinically non visible pre-lesions would be beneficial for the patient.

Therefore, it is an object of the present invention to provide alternative treatments for actinic keratosis with improved patient convenience.

This object is solved by claim 1 of the present invention. Accordingly, the use of molecules of the structure R₁-[O—CR₂R₃—CR₄R₅]ₙ—OH for the treatment of actinic keratosis is provided, whereby

R₁ is alkyl, long-chain alkyl, cycloalkyl, halogenalkyl,

R₂ to R₅ are independently from each other hydrogen, alkyl, alkoxy, cycloalkyl, halogenalkyl, aryl or halogenaryl, whereby in case n>1 every R along the carbon chain may differ from each other

n is an integer from 1 to 30

whereby for any R at suitable residues one or more CH₂-groups may independently from each other substituted by —O—, —S—, —NH—, —NR°—, —SiR° R°°—, —CO—, —COO—, —OCO—, —OCO—O—, —SO₂—, —S—CO—, —CO—S—, —CY¹=CY² oder —C≡C— in that way that O and/or S atoms are not directly bound to each other; terminal CH₃-groups are understood as CH₂—H groups.

Surprisingly it has been found that compounds of this structure can be used for the treatment of actinic keratosis. Without being bound to any theory the inventors believe that this at least partly due to a multiple synergistic effect since many compounds of these structure have found to have at least two of the following effects:
keratinolytic activity
analgetic activity

The profound but nevertheless gently keratinolytic activity is responsible for the primary curing effect. The analgetic activity significantly reduces the patients discomfort typically associated during repetitive treatment.

Generic group definition: Throughout the description and claims generic groups have been used, for example alkyl, alkoxy, aryl. Unless otherwise specified the following are preferred groups that may be applied to generic groups found within compounds disclosed herein:

alkyl: linear and branched C1-C8-alkyl,

long-chain alkyl: linear and branched C5-C20 alkyl

alkenyl: C2-C6-alkenyl,

cycloalkyl: C3-C8-cycloalkyl,

alkoxy: C1-C6-alkoxy,

long-chain alkoxy: linear and branched C5-C20 alkoxy

aryl: selected from homoaromatic compounds having a molecular weight under 300,

halogen: selected from the group consisting of: F; Cl; Br and I, halogenalkyl: selected from the group consisting of mono, di, tri-, poly and perhalogenated linear and branched Cl-C8-alkyl

Unless otherwise specified the following are more preferred group restrictions that may be applied to groups found within compounds disclosed herein:

alkyl: linear and branched C1-C6-alkyl, more preferred methyl, ethyl, propyl, isopropyl, buyl, isobutyl

long-chain alkyl: linear and branched C8-C15 alkyl, preferably linear C10-C12 alkyl

alkenyl: C3-C6-alkenyl,

cycloalkyl: C6-C8-cycloalkyl,

alkoxy: CI-C4-alkoxy,

long-chain alkoxy: linear and branched C5-C10 alkoxy, preferably linear C6-C8 alkoxy

aryl: selected from group consisting of: phenyl; biphenyl; naphthalenyl; anthracenyl; and phenanthrenyl,

halogen: selected from the group consisting of: F and Cl,

Synthesis methods and instructions of the molecules of the present invention are well-known in the art. In case that R₂ to R₅ are hydrogen (or only one of the R₂ to R₅ is alkyl) and that R₁ is alkyl or long-chain alkyl, the molecules can be synthesized from R₁—OH and either ethylene or propylene oxide.

According to an embodiment of the invention it is preferred that n is 7 to 11, preferably 8 to 10.

According to an embodiment of the invention it is preferred that R₁ is long-chain alkyl.

According to a preferred embodiment of the present invention R₂ to R₅ are hydrogen or only one of the R₂ to R₅ is alkyl, preferably methyl, the others are hydrogen.

Preferably the molecules according to the present invention comprise Polidocanol.

The term “Polidocanol” in the sense of the present invention especially relates to and/or includes oligopolyethoxylated dodecanol and/or comprises and/or includes the following chemical structure:

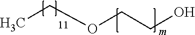

wherein the average m is 9 and the average molecular weight is around 600 Da (in case m is exactly 9 the molecular weight will be 582). Polidocanol can e.g. be made by oligoethoxylation of dodecanol with ethoxylating agents such as ethylene oxide.

Application of a formulation comprising the molecules of the present invention can be carried out by topical application or any form of injection, in particular by epicutaneous injection.

The molecules according to the present invention can be applied topically. Examples for a topic application of a formulation comprising a molecule according to the present invention include a cream, a patch, a salve, a gel, a powder, a dressing, ointment, microemulsions, liposomes, nanoparticles, semi-solid-nanoparticles without or with the use of iontophoresis or transdermal systems.

Preferably the concentration of the molecules according to the present invention is from about 0.001% to about 20% (wt/wt), preferred 0.1% to 10% and more preferred 1%-10%.

Alternatively, the molecules according to the present invention can be applied by epicutaneous injection. Examples for epicutaneous injection include aqueous solutions, suspensions, oily solutions, emulsions, microemulsions, liposomes, microspheres, nanoparticles and implants. The advantage of cutaneous injections (preferentially into actinic lesion) is the rapid onset of action and that the cytolytic effect is restricted to the targeted tissue. In general by local dermal application the systemic availability of compounds over time is reduced, since drug absorption from epidermal tissue is slow and sustained.

Preferably, each topical application or injection unit of the formulation has a distinct dose of the molecules according to the present invention according to the invention. This dose can reach from about 3 μMol to about 50 mMol, preferred from about 100 μMol to about 10 mMol, per volume of one application.

In case that the application occurs via injection, preferably one injection shot is sized from about 0.10 ml of the formulation to about 5.0 ml, more preferable between 0.5 and 2 ml, preferably around 1 ml.

In case that the application occurs via topical application, preferably per cm² treated area about 0.1-5 mg of the formulation, more preferable between 0.5-2 mg, preferably around 1 mg is applied.

The present invention furthermore relates to a formulation comprising at least one molecule according to the present invention of the structure R₁-[O—CR₂R₃—CR₄R₅]ₙ—OH for the treatment of actinic keratosis.

The present invention furthermore relates to process, comprising: administering molecules according to the present invention of the structure R₁-[O—CR₂R₃—CR₄R₅]ₙ—OH to a human in an amount effective for treating actinic keratosis

In an additional embodiment the claimed molecules are combined with an additional keratolytic agent in order to support the therapeutic effect. The keratolytic agent could simply be a chemical peel like compound like salicylic-acid ore could have a more efficient keratino-cytolic activity like retinoids.

Additional details, characteristics and advantages of the object of the invention are disclosed in the subclaims and the following description of the respective figure and example.

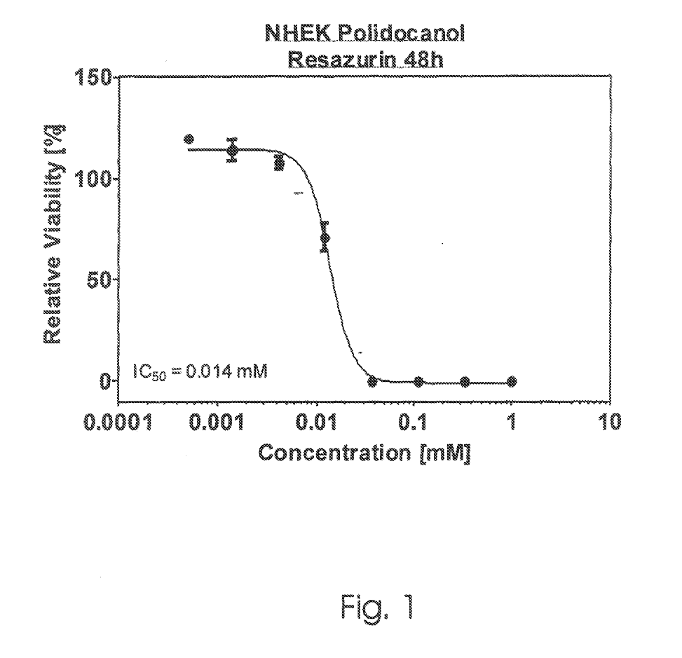

FIG. 1 is a diagram showing the relative viability vs. the concentration for the compound according to Example 1 of the present invention on NHEK-cells

### EXAMPLE 1

Example 1 refers to Polidocanol, whose structure is given above.

By using primary, normal human epidermal keratinocytes (NHEK, FIG. 1) the cytolytic potential of the drug compound was investigated.

The viability of the cells was monitored by their metabolic activity using the Resazurin-assay. Eight different concentrations (up to 1 mM) of polidocanol were investigated. Viability of the cells was plotted against the different concentration of test compound.

From the resulting sigmoid-curve the IC50 value for the cytolytic effect was determined and given in the respective figure. FIG. 1 depicts the relative viability of NHEK-cells vs. the concentration of test-compound using metabolic activity as read-out. The obtained data show a clear negative-impact of suggested drug compound on the viability of the cells.

The particular combinations of elements and features in the above detailed embodiments are exemplary only; the interchanging and substitution of these teachings with other teachings in this and the patents/applications incorporated by reference are also expressly contemplated. As those skilled in the art will recognize, variations, modifications, and other implementations of what is described herein can occur to those of ordinary skill in the art without departing from the spirit and the scope of the invention as claimed. Accordingly, the foregoing description is by way of example only and is not intended as limiting. In the claims, the word “comprising” does not exclude other elements or steps, and the indefinite article “a” or “an” does not exclude a plurality. The mere fact that certain measures are recited in mutually different dependent claims does not indicate that a combination of these measured cannot be used to advantage. The invention's scope is defined in the following claims and the equivalents thereto. Furthermore, reference signs used in the description and claims do not limit the scope of the invention as claimed.

1-7. (canceled)

8. A method of treating actinic keratosis in a subject in need thereof, comprising administration of an effective amount of a compound of the following structure
R₁-[O—CR₂R₃—CR₄R₅]ₙ—OH wherein
n is an integer from 1 to 30;
R₁ is alkyl long-chain alkyl, cycloalkyl, halogenalkyl; and
R₂ to R₅ are independently from each other hydrogen, alkyl, alkoxy, cycloalkyl, halogenalkyl, aryl or halogenaryl, wherein when n>1 every R₂ to R₅ along the carbon chain may differ from each other,
wherein for any R at suitable residues one or more CH₂-groups may independently from each other substituted by —O—, —S—, —NH—, —NR°—, —SiR°R°°—, —CO—, —COO—, —OCO—, —OCO—O—, —SO₂—, —CO—S—, —CY¹=CY² or C≡C—, it being understood that O and/or S atoms are not directly bound to each other: and terminal CH₃-groups are understood as meaning CH₂—H groups.

9. The method according to claim 8, wherein n is 7 to 11.

10. The method according to claim 8, wherein n is 8 to 10.

11. The method according to claim 8, wherein R₂ to R₅ are hydrogen or one of R₂ to R₅ is alkyl and the others are hydrogen.

12. The method according to claim 11, wherein one of R₂ to R₅ is methyl and the others are hydrogen.

13. The method according to claim 8, wherein the compound is polidocanol.

14. The method according to claim 8, wherein the compound is administered topically or in form of an injection.

15. The method according to claim 8, wherein the compound is administered at a dose between from about 3 μMol to about 50 mMol.
